(19) United States
(12) Patent Application Publication
(10) Pub. No.: US 2010/0316753 A1
(43) Pub. Date: Dec. 16, 2010

(54) FORMULATIONS AND METHODS FOR REDUCING ABDOMINAL FAT AND WAIST CIRCUMFERENCE

(76) Inventor: Harunobu Amagase, Phoenix, AZ (US)

Correspondence Address: QUARLES & BRADY LLP, 411 E. WISCONSIN AVENUE, SUITE 2040, MILWAUKEE, WI, 53202-4497, US

(21) Appl. No.: 12/864,626

(22) PCT Filed: Jan. 26, 2009
(86) PCT No.: PCT/US09/31992

§ 371 Date: Jul. 26, 2010

Related U.S. Application Data
(60) Provisional application No. 61/024,831, filed on Jan. 30, 2008.

(51) Int. Cl. A61K 36/815; A61P 3/04

(52) U.S. Cl. 424/777; 424/725

(57) ABSTRACT

The invention provides methods for reducing abdominal fat and waist circumference in a human subject by administering Lycium plants or Lycium plant extract preparations, preferably goji juice. The invention also provides a food or dietary supplement formulation which contains Lycium plants or Lycium plant extract preparations.

### FIELD OF THE INVENTION

The invention relates generally to formulations and methods for reducing abdominal fat and reducing waist circumference in human subjects.

### BACKGROUND OF THE INVENTION

Excess abdominal fat is a strong risk factor for heart attacks, strokes and other diseases. Based on new research, doctors pay more attention to patients' waist circumferences than their body-mass-index (BMI). Risk begins to mount at a waist size above 31 inches for women and 37 inches for men; measurements above 35 inches for women and 40 inches for men are considered to be in the danger range.

Methods for reducing abdominal fat vary from surgery to changing one's diet. However, surgery for “spot reducing” is not considered a long term solution to reduce one's waist size, and therefore one's disease risk. Even liposuction does not remove fat from the abdominal organs, where it is most dangerous. Many people are advised to eat less, to avoid calorie-dense foods that are high in fat and/or sugar, and to increase their intake of foods such as fruits, vegetables and whole grains, which are filling but not fattening. Physically active tasks should also be incorporated into the daily life.

However, while many people have heard the above suggestions from doctors and physical practitioners, it is often difficult to change one's life style and as a result, the numbers of obese subjects is rising sharply. Accordingly, a need exists for compositions and methods of reducing abdominal fat in subjects which avoid invasive and rigorous intervention.

### SUMMARY OF THE INVENTION

Here, the inventors demonstrate versatile formulations and methods for reducing abdominal fat and waist circumference in human subjects.

Accordingly, the present invention provides a method for reducing abdominal fat in a subject, the method comprising administering a composition comprising Lycium plants (known as goji, gouqizi, Fructus lycii or wolfberry) or Lycium plant extract preparations to the subject, whereby the subject's abdominal fat is reduced. In an alternate preferred embodiment, the composition comprises goji juice.

In another embodiment, the invention provides a method of reducing the waist circumference in a subject, the method comprising administering a composition comprising Lycium plants or Lycium plant extract preparations, whereby the subject's waist circumference is reduced. In an alternate preferred embodiment, the composition comprises goji juice.

In yet another embodiment, the invention provides a food formulation comprising an effective amount of Lycium plants or Lycium plant preparations and at least one other food ingredient. In an alternate preferred embodiment, the composition comprises goji juice.

The invention also encompasses the use of Lycium plants or Lycium plant preparations, preferably goji juice, for the manufacture of food formulations to reduce abdominal fat and waist circumference in a subject ingesting the food formulation. Lycium plants or Lycium plant preparations, preferably goji juice, packaged and presented for use in the reduction of abdominal fat and waist circumference are also within the invention.

Other objects, features and advantages of the present invention will become apparent after review of the specification, claims and drawings.

### BRIEF DESCRIPTION OF THE DRAWINGS

FIG. 1. Changes of parametric markers on Day 15 after 14 days goji consumption.

FIG. 2. Waist circumference changes in human subjects during and after the 14-days test period.

### DETAILED DESCRIPTION OF THE INVENTION

### I. In General

Before the present materials and methods are described, it is understood that this invention is not limited to the particular methodology, protocols, materials, and reagents described, as these may vary. It is also to be understood that the terminology used herein is for the purpose of describing particular embodiments only, and is not intended to limit the scope of the present invention which will be limited only by any later-filed nonprovisional applications.

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, and “having” can be used interchangeably.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications and patents specifically mentioned herein are incorporated by reference for all purposes including describing and disclosing the chemicals, instruments, statistical analysis and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

### II. The Invention

Herein, the inventors provide formulations and methods for reducing abdominal fat and reducing waist circumference in human subjects.

The Lycium plants in this invention are the plants belonging to the family of solanaceous defoliated shrubbery, such as, for example, Lycium barbarum and Lycium chinense. The most suitable plant for this invention is Lycium barbarum (known as goji, gouqizi, Fructus lycii, or wolfberry). The preferred portion for this invention is the fruit of this plant. Leaf, root or stem may also be utilized for this invention. These materials can be processed as juice or dried by processing and/or extraction methods commonly known in the art.

In addition, the materials derived from the cell culture of the plants can also be utilized as materials for this invention. The juice or extract of the Lycium plants in this invention is preferably the preparation made from plant materials prepared or extracted from water or alcohol. The Lycium plants for the preparation or extraction can be squeezed or crushed with or without a moderate temperature to effect extraction efficiency, as is routinely understood in the art. It is also possible to crush and homogenize the plants to make the juice after separating skin, seeds and other parts. The extract prepared for dietary intake can be utilized as is, in concentrated fluid or powder form after concentration under vacuum or lyophilization.

The administrative dosage of the preparation effective to reduce abdominal fat and waist circumference is varied by the age, body weight or body condition of the human subject. It is recommended to take orally 0.1 oz (3 ml) through 33 oz (990 ml) in a day by adults as a liquid preparation of Lycium plants or extracted Lycium plants. The more feasible and preferred dosage ranges approximately from 1 oz (30 ml) to 8 oz (240 ml) of the preparations per day for an adult human.

The following examples describing materials and methodology are offered for illustrative purposes only, and are not intended to limit the scope of the present invention.

### III. Examples

Example 1. Controlled Study

This example describes a controlled study which examined the efficacy of orally consumed goji berry, Lycium barbarum, as a standardized juice (GoChi) in healthy adults on waist circumference after consuming 120 ml (4 oz) of GoChi/day for 14 days. All subjects were requested to perform 15 minutes walk as exercise and no food or snack after 7 pm during the test period. GoChi was separately consumed 90 ml (3 oz) in the morning and 30 ml (1 oz) right before the bed time. Data were statistically analyzed for changes between day 1 and day 15.

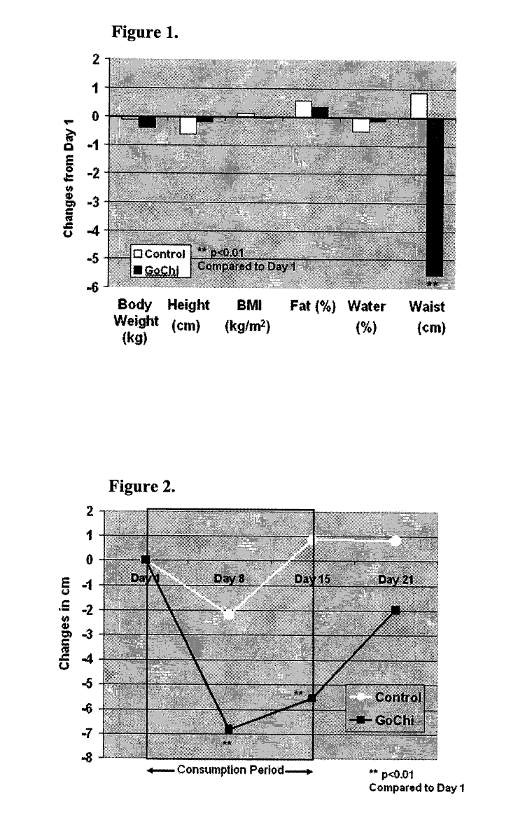

Waist circumference in GoChi group (N=14) on day 15 has been reduced significantly by 5.54 cm from day 1 (p<0.01). All other parametric data (body weight, etc.) were not significantly different between groups or between day 1 and day 15 for either group. In contrast, the placebo group (N=14) showed no significant changes in any parameters as shown in FIG. 1. This reduction in waist circumference was caused within 8 days after the commencement of the GoChi intake and kept in a similar level throughout the 14-day administration period (FIG. 2).

These results clearly indicate that daily consumption of GoChi for 14 days reduces waist circumference related to abdominal fat.

Other embodiments and uses of the invention will be apparent to those skilled in the art from consideration from the specification and practice of the invention disclosed herein. All references cited herein for any reason, including all journal citations and U.S./foreign patents and patent applications, are specifically and entirely incorporated herein by reference.

It is understood that the invention is not confined to the specific reagents, formulations, reaction conditions, etc., herein illustrated and described, but embraces such modified forms thereof as come within the scope of the following claims.

1. A method of reducing abdominal fat in a subject, comprising administering a composition comprising Lycium plants or Lycium plant extract preparations to the subject, whereby the subject's abdominal fat is reduced.

2. The method of reducing abdominal fat in a subject according to claim 1, wherein said composition is goji juice.

3. A method of reducing the waist circumference in a subject, comprising administering a composition comprising Lycium plants or Lycium plant extract preparations to the subject, whereby the subject's waist circumference is reduced.

4. The method of reducing the waist circumference in a subject according to claim 3, wherein said composition is goji juice.

5. A food formulation comprising an effective amount of Lycium plants or Lycium plant preparations to reduce waist circumference in a subject ingesting said food formulation and at least one other food ingredient.

6. A The food formulation according to claim 5, wherein said plant preparations comprise an effective amount of goji juice to reduce waist circumference in a subject ingesting said food or dietary supplement formulation.

7. A food or dietary supplement formulation comprising an effective amount of Lycium plants or Lycium plant preparations to reduce abdominal fat in a subject ingesting said food or dietary supplement formulation and at least one other food or dietary supplement ingredient.

8. The food or dietary supplement formulation according to claim 7, wherein said plant preparations comprise an effective amount of goji juice to reduce abdominal fat in a subject ingesting said food or dietary supplement formulation.

9. (canceled)

10. (canceled)

11. (canceled)

12. (canceled)

13. (canceled)

14. (canceled)

15. (canceled)

16. (canceled)
